(19) United States
(12) Patent Application Publication
(10) Pub. No.: US 2011/0136911 A1
(43) Pub. Date: Jun. 9, 2011

(54) TREATMENT OF SEPSIS AND INHIBITION OF MIF BY D-T4

(75) Inventor: Yousef Al-Abed, Locust Valley, NY (US)

(73) Assignee: The Feinstein Institute for Medical Research, Manhasset, NY (US)

(21) Appl. No.: 12/735,161

(22) PCT Filed: Dec. 18, 2008
(86) PCT No.: PCT/US2008/013839

§ 371 Date: Sep. 8, 2010

Related U.S. Application Data
(60) Provisional application No. 61/008,600, filed on Dec. 20, 2007.

(51) Int. Cl. A61K 31/198; C07C 229/36; A61P 29/00; A61P 7/00; A61P 37/06; A61P 31/04; A61P 3/10; A61P 17/06; A61P 9/00; A61P 31/14; A61P 25/28; A61P 9/10; A61P 37/08; A61P 31/22; A61P 31/18; A61P 33/06; A61P 17/00; A61P 17/02; A61P 11/00

(52) U.S. Cl. 514/567; 562/444

(57) ABSTRACT

Methods and compositions are disclosed for the use of dextrothyroxine (D-T4) to treat sepsis, inflammation, and conditions and diseases in which it is desirable to inhibit macrophage migration inhibitory factor (MIF).

### CROSS-REFERENCE TO RELATED APPLICATION

This application claims the benefit of U.S. Provisional Patent Application No. 61/008,600, filed on Dec. 20, 2007, the content of which is hereby incorporated by reference into the subject application.

### FIELD OF THE INVENTION

The present invention relates to uses of dextrothyroxine (D-T4) to treat sepsis, inflammation, and diseases and conditions that can be treated by inhibiting macrophage migration inhibitory factor (MIF).

### BACKGROUND OF THE INVENTION

Throughout this application various publications are referred to in parenthesis. Citations for these references may be found at the end of the specification immediately preceding the claims. The disclosures of these publications are hereby incorporated by reference in their entireties into the subject application to more fully describe the art to which the subject application pertains.

Sepsis is a potentially lethal systemic inflammatory reaction to infection that affects approximately 750,000 people and kills more than 215,000 people annually at a national cost of $16.7 billion in the United States alone (Martin et al. 2003). Annual deaths from sepsis equal the number from myocardial infarction (Agnus et al. 2001). Cardiovascular dysfunction is a common sequelae of severe sepsis. Sepsis is mediated, at least in part, by blood-borne cytokines. Among these, macrophage migration inhibitory factor (MIF) has been shown to play a critical role in the pathogenesis of this condition (Beishuizen et al. 2001; Calandra et al. 2000, 2001; Lue et al. 2002), mediating both cardiac dysfunction (Garner et al. 2003, Sakuragi et al. 2007) and mortality (Al-Abed et al. 2005, Lin et al. 2005, Monig et al. 1999, Sakuragi et al. 2007). Released from the lung during sepsis (Lin et al. 2005, Sakuragi et al. 2007), MIF is a potent pro-inflammatory cytokine, acting in autocrine and paracrine pathways to activate macrophages (Al-Abed et al. 2005) and cardiomyocytes (Lin et al. 2005) and counteract glucocorticoid effects (Calandra et al. 1995).

MIF is produced by numerous cell types, including immune and endocrine cells, and is recognized as a pro-inflammatory counter-regulator of the anti-inflammatory activities of the glucocorticoids. In vitro, MIF expression abrogates the anti-inflammatory and immunosuppressive effect of glucocorticoid production on pro-inflammatory cytokines (TNF-α, IL-1, IL-2, IL-6, and IL-8) (Calandra and Bucala, 1997; Donnelly et al., 1997). In mice, administration of recombinant MIF, together with dexamethasone, completely blocks the protective effects of dexamethasone on lipopolysaccharide (LPS) lethality (Calandra et al. 1995). MIF is critically involved in the pathogenesis of a variety of inflammatory diseases. In particular, animal models of Gram-positive, Gram-negative, and polymicrobial sepsis, as well as MIF knockout models, indicate a critical role of MIF in sepsis (Calandra et al., 2000; Bozza et al., 1999; Bernhagen et al., 1993).

In vivo studies demonstrate that MIF is an important late-acting mediator of systemic inflammation. Deletion of the MIF gene in mice conferred protection against lethal endotoxemia and staphylococcal toxic shock (Bozza et al., 1999). In addition, administration of neutralizing anti-MIF antibody protected mice from: (a) LPS-induced lethality; (b) lethal peritonitis and septic shock induced by E. coli peritonitis and (c) fulminant septic shock induced by cecal ligation and puncture (CLP) in TNF-α deficient mice (Calandra, 2001; Bernhagen et al., 1993). In contrast to early mediators such as TNF-α and IL-1β, the appearance of MIF in the blood peaks and then plateaus later after the onset of CLP, thus indicating a longer window of opportunity for therapeutic treatment. Consequently, anti-MIF therapies are potentially more beneficial than anti-TNF-α and anti-IL-1 therapies, which have demonstrated limited benefits for patients with severe sepsis (Abraham, 1999). In contrast, administration of anti-MIF antibody 8 hours post-induction of sepsis confers significant protection in a murine CLP model of sepsis versus animals receiving control IgG. Human studies also support a role for MIF in septic shock (Beishuizen et al., 2001; Calandra et al., 2000). A correlation has been documented between the severity of injury or infection in trauma patients and MIF levels in the serum, with increased circulating levels of MIF displayed in patients with severe sepsis (6-fold) and in patients with septic shock (15-fold) (Calandra et al., 2000).

Studies examining cardiac function during sepsis have identified MIF as a myocardial depressant factor, and anti-MIF antibody administration significantly improves cardiac performance during septic shock (Chagnon et al. 2005, Garner et al. 2003, Lin et al. 2005, Willis et al. 2005). MIF accumulates within the lung during sepsis and the lung then acts as a major source of the MIF released into the pulmonary circulation simultaneous with the onset of cardiac dysfunction. Additionally, MIF is a late mediator of sepsis and a critical factor in the pathophysiology of sepsis (Al-Abed et al. 2005, Sakuragi et al. 2007). Three-dimensional X-ray crystallography of MIF shows that the molecule exists as a homotrimer (Kato et al. 1996; Lolis et al. 1996; Lubetsky et al. 1999, 2002; Subramanya et al. 1996; Sugimoto et al. 1995, 1996; Sun et al. 1996; Suzuki et al. 1994, 1996; Taylor et al. 1998, 1999) and the hydrophobic pocket formed between adjacent monomers has been shown to be important for the inflammatory activity of MIF (Al-Abed et al. 2005, Cvetkovic et al. 2005, Dios et al. 2002, Lubetsky et al. 2002, Nicoletti et al. 2005, Sakuragi et al. 2007, Senter et al. 2002). MIF can catalyze the tautomerization of dopachrome methyl esters into their corresponding indole derivatives (Rosengren et al., 1996), although the parameters for this reaction indicate that it is unlikely to be a natural function of MIF (Rosengren et al. 1996, Suzuke et al. 1996).

Small molecules that bind to the tautomerase active site of MIF inhibit its pro-inflammatory activity and increase survival in severe sepsis. For example, the compound (S,R)-3-(4-hydroxyphenyl)-4,5-dihydro-5-isoxazole acetic acid methyl ester (ISO-1) was recently designed as an inhibitor of MIF activity (PCT International Patent Publication No. WO 02/100332). ISO-1 fits into the hydrophobic active site of MIF, an interaction confirmed by the crystal structure of the MIF complex with ISO-1. ISO-1 binding to this tautomerase active site abolishes the inflammatory ability of MIF. ISO-1 significantly inhibits MIF proinflammatory activities in vitro (Al-Abed et al. 2005, Lubetsky et al. 2002) and significantly improves both cardiac function (Sakuragi et al. 2007) and long-term survival in animal models of polymicrobial sepsis (Al-Abed et al. 2005).

The thyroid gland is the source of iodothyronine hormones including thyroxine (T4) and 3,5,3′-triiodothyronine (T3) (FIG. 1). These hormones are essential for normal growth and development and play an important role in energy metabolism. Most of the organic iodine is in the form of T4 (90-95%), while triiodothyronine represents a relatively minor fraction (about 5%). The thyroid hormones are transported in the blood in strong but non-covalent association with certain plasma proteins. Thyroxine-binding globulin is the major carrier of thyroid hormones and it binds one molecule of T4 per molecule of protein with a very high affinity (Ka is about 10⁻¹⁰ M). Triiodothyronine is bound less avidly. T4, but not triiodothyronine, is bound by transthyretin (also called thyroxine-binding prealbumin). Transthyretin has four apparently identical subunits, but has only a single high affinity-binding site. Thyroxine also binds to the apolipoproteins of the high density lipoprotein, HDL2 and HDL3, the biological significance of which remains unclear (Benvenga et al. 1992).

Binding of thyroid hormones to plasma proteins protects the hormones from metabolism and excretion, resulting in long half-lives in the circulation. The free (unbound) hormone is a small percentage (about 0.03% of thyroxine and 0.3% of triiodothyronine) of the total hormone in plasma (Larsen et al. 1981a,b). The “free hormone” concept is essential to understanding the regulation of thyroid function: only the unbound hormone has metabolic activity (Mendel 1989).

Disorders of the thyroid are common, and effective treatments are available. There is a vast literature available on the changes in thyroid function that occur during non-thyroidal illness. However, thyroid hormone therapy in humans is still controversial for the treatment of the “low T4 syndrome or low T3 syndrome” (euthyroid sick syndrome (ESS)) that is the result of a non-thyroidal illness (Brent and Hershman 1986). The ESS term describes abnormalities in thyroid function usually observed in critically ill patients including septic patients admitted to the intensive care unit (ICU) (Brent and Hershman 1986, Leon-Sanz et al. 1997). In septic patients, all thyroid hormones and the thyroid stimulating hormone (TSH) are already markedly decreased on the day of admission to the ICU (Baue et al. 1984, Brent and Hershman 1986, Monig et al. 1999, Phillips et al. 1984, Slag et al. 1981). The syndrome is characterized by low free and total T4 and T3 and only survivors present a significant increase in T4 and T3.

In septic patients with poor prognosis, circulating free thyroid hormone T4 concentrations typically fall while MIF levels increase (Baue et al. 1984, Beisswenger et al. 1995, Brent and Hershman 1986, Calandra et al. 2000, Leon-Sanz et al. 1997, Monig et al. 1999, Phillips et al. 1984, Slag et al. 1981). In contrast, septic patients with better prognosis typically maintain or increase free T4 levels. T4 administration in rat models of sepsis improves the survival rate and restores the level of circulating free T4 (Inan et al. 2003). In contrast, Little (1985) reported that T4 administration caused increased mortality to rats infected with Streptococcus pneumoniae.

Dextrothyroxine (D-T4) is the stereoisomer of L-T4 (FIG. 1). T4 and D-T4 behave similarly in some biological systems (Benvenga et al. 1989, Pizzagalli et al. 2002, Yamamoto et al. 2000). However, there are many differences in biological activity between T4 and D-T4 (Goncalves et al. 1990; Kavok et al. 2001; Lawrence et al. 1989; Lin et al. 1994, 1996; Neves et al. 2002; Yan and Hinkle 1993; Yosha et al. 1984). Based on the numerous differences in biological activity between T4 and D-T4 and on the report of Little (1985), a therapeutic effect of D-T4 in treating sepsis, as well as other disorders, would be unexpected.

### SUMMARY OF THE INVENTION

The invention provides a method for treating sepsis and/or septic shock in a subject comprising administering dextrothyroxine (D-T4) to the subject in an amount effective to treat sepsis and/or septic shock.

The invention also provides a method of treating a subject having a condition or disease in which it is desirable to inhibit macrophage migration inhibitory factor (MIF), the method comprising administering to the subject an amount of dextrothyroxine (D-T4) effective to inhibit MIF.

The invention further provides a method for reducing the pathogenic consequences of an inflammatory condition or an inflammatory cytokine cascade or for treating an inflammatory disease or condition in a subject comprising administering dextrothyroxine (D-T4) to the subject in an amount effective to reduce the pathogenic consequences of an inflammatory condition or an inflammatory cytokine cascade or to treat an inflammatory disease or condition.

The invention provides pharmaceutical compositions comprising dextrothyroxine (D-T4) formulated in dosage form for treating sepsis and/or septic shock, for treating a condition or disease in a subject in which it is desirable to inhibit macrophage migration inhibitory factor (MIF), for reducing the pathogenic consequences of an inflammatory condition or an inflammatory cytokine cascade and for treating an inflammatory disease or condition.

The invention still further provides a method of preparing a pharmaceutical composition for treating sepsis and/or septic shock, the method comprising formulating dextrothyroxine (D-T4) in a pharmaceutical composition in an amount effective to treat sepsis and/or septic shock.

The invention also provides a method of preparing a pharmaceutical composition for treating a condition or disease in a subject in which it is desirable to inhibit macrophage migration inhibitory factor (MIF), the method comprising formulating dextrothyroxine (D-T4) in a pharmaceutical composition in an amount effective to inhibit MIF.

The invention further provides a method of preparing a pharmaceutical composition for reducing the pathogenic consequences of an inflammatory condition or an inflammatory cytokine cascade or for treating an inflammatory disease or condition, the method comprising formulating dextrothyroxine (D-T4) in a pharmaceutical composition in an amount effective to reduce the pathogenic consequences of an inflammatory condition or an inflammatory cytokine cascade or treat an inflammatory disease or condition.

### BRIEF DESCRIPTION OF THE DRAWINGS

FIG. 1. Structure of 3,5,3′-triiodothyronine (T3), L-thyroxine (T4) and D-thyroxine (D-T4).

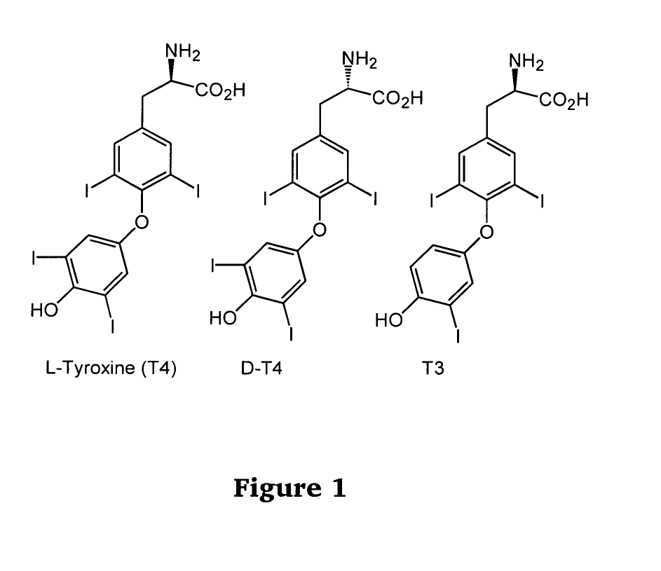

FIG. 2. Dose-dependent inhibition of the tautomerase enzymatic activity of MIF by T4, D-T4, T3 and ISO-1.

FIG. 3. D-T4 is protective after 24 h late treatment in a cecal ligation and puncture (CLP) model of sepsis. Mice were injected intraperitoneally with D-T4 (∘; 4 mg/kg, n=10) or vehicle (, n=10) 24 hours after CLP. Additional two injections were given on day 2 and day 3.

FIG. 4. Repeat of the cecal ligation and puncture experiment with a second group of animals showing that D-T4 dramatically increases survival in this model.

FIG. 5. D-T4 is a potent inhibitor of tumor necrosis factor alpha (TNFα) secretion from lipopolysaccharide (LPS)-stimulated RAW macrophages.

FIG. 6. D-T4 reduces an inflammatory cytokine cascade-induced inflammatory response in wild-type but not in MIF knockout mice (−/−) in a skin pouch model of acute inflammation. The plot shows the number of infiltrating cells normalized as a percent of the corresponding animals that received vehicle alone (veh.). *p<0.04 relative to vehicle alone; n.s., not significant.

### DETAILED DESCRIPTION OF THE INVENTION

The invention provides a method for treating sepsis and/or septic shock in a subject comprising administering dextrothyroxine (D-T4) to the subject in an amount effective to treat sepsis and/or septic shock.

Sepsis can be characterized as an inflammatory state caused by infection. It is a toxic condition resulting from the spread of bacteria or their products from a focus of infection. Septicemia (infection in the blood) is a subset of sepsis. Critical forms of sepsis include severe sepsis with acute organ dysfunction and septic shock with refractory arterial hypotension. Septic shock can be a life-threatening form of sepsis that typically results from gram-negative bacteria and their toxins in the bloodstream.

As used herein, to treat sepsis means to prevent or reduce a physiological effect of sepsis. Preferably, treatment prevents or reduces serum elevation of TNF-α. Preferably, treatment prevents or reduces tissue and/or organ injury in the subject. Preferably, the treatment prevents or reduces septic shock. Preferably, treatment improves survival of the subject. Preferably, treatment inhibits macrophage migration inhibitory factor (MIF).

Preferably, the methods of the present invention prevent or reduce one or more physiologic effect of sepsis, including shock (which in turn affects endothelial cell function, smooth muscle contractility, cardiac output, stroke volume, systemic oxygen delivery, lactic acidosis, hemoconcentration, total peripheral vascular resistance and/or regional blood perfusion), renal function, hepatic function, gut absorptive function, adrenal function, insulin responsiveness, altered cytokine (e.g., HMGB1, IL-10, TNF-α, IL-1β and/or IL-6) release or appearance, and physiological effects of altered cytokine release (e.g., inflammation). To evaluate the prevention or reduction of physiologic effects of sepsis, it is preferred that physiologic effects that are easily measured are compared before and after treatment. In a preferred embodiment, the measured physiological effect of the sepsis is elevation of serum TNF-α levels. Determination of shock, or its direct effects (e.g., hemoconcentration, peripheral vascular resistance, etc.) is also easily measured and can be utilized.

The invention also provides a pharmaceutical composition comprising dextrothyroxine (D-T4) formulated in dosage form for treating sepsis and/or septic shock.

The invention further provides a method of preparing a pharmaceutical composition for treating sepsis and/or septic shock, the method comprising formulating dextrothyroxine (D-T4) in a pharmaceutical composition in an amount effective to treat sepsis and/or septic shock.

The invention is also directed to a method of treating a subject having a condition or disease in which it is desirable to inhibit macrophage migration inhibitory factor (MIF), the method comprising administering to the subject an amount of dextrothyroxine (D-T4) effective to inhibit MIF.

The subject can have or be at risk for a condition or disease that comprises an inflammatory cytokine cascade that is at least partially mediated by MIF. Examples of such conditions or diseases include, but are not limited to, proliferative vascular disease; acute respiratory distress syndrome; cytokine-mediated toxicity; psoriasis; interleukin-2 toxicity; appendicitis; peptic, gastric and/or duodenal ulcer; peritonitis; pancreatitis; ulcerative, pseudomembranous, acute and ischemic colitis; diverticulitis; epiglottitis; achalasia; cholangitis; cholecystitis; hepatitis; inflammatory bowel disease; Crohn's disease; enteritis; Whipple's disease; asthma; allergy; anaphylactic shock; immune complex disease; organ ischemia; reperfusion injury; organ necrosis; hay fever; sepsis; septicemia; endotoxic shock; cachexia; hyperpyrexia; eosinophilic granuloma; granulomatosis; sarcoidosis; septic abortion; epididymitis; vaginitis; prostatitis; urethritis; bronchitis; emphysema; rhinitis; cystic fibrosis; pneumonitis; alvealitis; bronchiolitis; pharyngitis; pleurisy; sinusitis; influenza; respiratory syncytial virus infection; herpes infection; HIV infection; hepatitis B virus infection; hepatitis C virus infection; disseminated bacteremia; Dengue fever; candidiasis; malaria; filariasis; amebiasis, hydatid cysts, burns, dermatitis, dermatomyositis, sunburn, urticaria, warts, wheals; vasulitis; angiitis; endocarditis; arteritis; atherosclerosis; thrombophlebitis; pericarditis; myocarditis; myocardial ischemia; periarteritis nodosa; rheumatic fever; Alzheimer's disease; coeliac disease; congestive heart failure; meningitis; encephalitis; multiple sclerosis; cerebral infarction; cerebral embolism; Guillame-Barre syndrome; neuritis; neuralgia; spinal cord injury; paralysis; uveitis; arthritides; arthralgias; osteomyelitis; fasciitis; Paget's disease; gout; periodontal disease; rheumatoid arthritis; synovitis; myasthenia gravis; thryoiditis; systemic lupus erythematosus; Goodpasture's syndrome; Behcets's syndrome; allograft rejection; graft-versus-host disease; ankylosing spondylitis; type 1 diabetes; type 2 diabetes; Berger's disease; Retier's syndrome and Hodgkins disease.

The subject can have or be at risk for an autoimmune disease. MIF has been shown to play an important role in autoimmune disease. See, e.g., Cvetjovic et al., 2005. Examples of autoimmune disease include, but are not limited to, multiple sclerosis, systemic lupus erythematosus, rheumatoid arthritis, graft-versus-host disease, autoimmune pulmonary inflammation, autoimmune encephalomyelitis, Guillain-Barre syndrome, autoimmune thyroiditis, insulin-dependent diabetes mellitus, Crohn's disease, scleroderma, psoriasis, Sjögren's syndrome and autoimmune inflammatory eye disease. The present methods would thus be useful in treatment of autoimmune disease.

The subject can have a tumor. MIF is known to promote tumor invasion and metastasis. See, e.g., Sun et al., 2005. The present methods would therefore be useful for treatment of a mammal that has a tumor.

The subject can have or be at risk for developing inflammation. Diseases involving inflammation include, for example, proliferative vascular disease, acute respiratory distress syndrome, cytokine-mediated toxicity, psoriasis, interleukin-2 toxicity, appendicitis, peptic, gastric and duodenal ulcers, peritonitis, pancreatitis, ulcerative, pseudomembranous, acute and ischemic colitis, diverticulitis, epiglottitis, achalasia, cholangitis, cholecystitis, hepatitis, inflammatory bowel disease, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, alvealitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thryoiditis, systemic lupus erythematosus, Goodpasture's syndrome, Behcets's syndrome, allograft rejection, graft-versus-host disease, ankylosing spondylitis, Berger's disease, type 1 diabetes, type 2 diabetes, Retier's syndrome, and Hodgkins disease.

These methods of the invention can be used on any mammal. Preferably, the mammal is a human.

The invention also provides a pharmaceutical composition comprising dextrothyroxine (D-T4) formulated in dosage form for treating a condition or disease in a subject in which it is desirable to inhibit macrophage migration inhibitory factor (MIF).

The invention further provides a method of preparing a pharmaceutical composition for treating a condition or disease in a subject in which it is desirable to inhibit macrophage migration inhibitory factor (MIF), the method comprising formulating dextrothyroxine (D-T4) in a pharmaceutical composition in an amount effective to inhibit MIF.

The invention provides a method for reducing the pathogenic consequences of an inflammatory condition or an inflammatory cytokine cascade or for treating an inflammatory disease or condition in a subject comprising administering dextrothyroxine (D-T4) to the subject in an amount effective to reduce the pathogenic consequences of an inflammatory condition or an inflammatory cytokine cascade or to treat an inflammatory disease or condition.

The invention also provides a pharmaceutical composition comprising dextrothyroxine (D-T4) formulated in dosage form for reducing the pathogenic consequences of an inflammatory condition or an inflammatory cytokine cascade or for treating an inflammatory disease or condition.

The invention further a provides a method of preparing a pharmaceutical composition for reducing the pathogenic consequences of an inflammatory condition or an inflammatory cytokine cascade or for treating an inflammatory disease or condition, the method comprising formulating dextrothyroxine (D-T4) in a pharmaceutical composition in an amount effective to reduce the pathogenic consequences of an inflammatory condition or an inflammatory cytokine cascade or treat an inflammatory disease or condition.

The pharmaceutical compositions further comprise a pharmaceutically acceptable carrier. By “pharmaceutically acceptable” it is meant a material that (i) is compatible with the other ingredients of the composition without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers include, without limitation, any of the standard pharmaceutical carriers such as phosphate buffered saline solutions, water, emulsions such as oil/water emulsions, microemulsions, and the like.

D-T4 can be formulated without undue experimentation for administration to a subject, including humans, as appropriate for the particular application. Additionally, proper dosages of D-T4 can be determined without undue experimentation using standard dose-response protocols.

Accordingly, the compositions designed for oral, lingual, sublingual, buccal and intrabuccal administration can be made without undue experimentation by means well known in the art, for example with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, cornstarch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

The compositions can easily be administered parenterally such as for example, by intravenous, intramuscular, intrathecal or subcutaneous injection. Parenteral administration can be accomplished by incorporating D-T4 into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering D-T4, in a pharmaceutical composition, into the rectum or large intestine. This can be accomplished using suppositories or enemas. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the composition through the skin. Transdermal formulations include patches (such as the well-known nicotine patch), ointments, creams, gels, salves and the like.

The compositions can also be prepared for nasal administration. As used herein, nasal administration includes administering D-T4 to the mucous membranes of the nasal passage or nasal cavity of the patient. Pharmaceutical compositions for nasal administration of the compound include therapeutically effective amounts of the compound prepared by well-known methods to be administered, for example, as a nasal spray, nasal drop, suspension, gel, ointment, cream or powder. Administration of the compositions may also take place using a nasal tampon or nasal sponge.

D-T4 may be administered per se or in the form of a pharmaceutically acceptable salt. When used in medicine, the salts should be both pharmacologically and pharmaceutically acceptable, but non-pharmaceutically acceptable salts may conveniently be used to prepare the free active compound or pharmaceutically acceptable salts thereof. Pharmacologically and pharmaceutically acceptable salts include, but are not limited to, those prepared from the following acids: hydrochloric, hydrobromic, sulphuric, nitric, phosphoric, maleic, acetic, salicyclic, p-toluenesulfonic, tartaric, citric, methanesulphonic, formic, malonic, succinic, naphthalene-2-sulphonic, and benzenesulphonic. Also, pharmaceutically acceptable salts can be prepared as alkaline metal or alkaline earth salts, such as sodium, potassium or calcium salts of the carboxylic acid group.

This invention will be better understood from the Experimental Details, which follow. However, one skilled in the art will readily appreciate that the specific methods and results discussed are merely illustrative of the invention as described more fully in the claims that follow thereafter.

### Experimental Details

### D-T4 and T4 Inhibit MIF Activity

Dopachrome Tautomerase Assay: L-Dopachrome methyl ester was prepared at 2.4 mM through oxidation of L-3,4-dihydroxyphenylalanine methyl ester with sodium periodate as previously described (Dios et al. 2002). Activity was determined at room temperature by adding dopachrome methyl ester (0.3 ml) to a cuvette containing 50 nM MIF in 50 mM potassium phosphate buffer, pH 6, 0.5 mM EDTA and measuring the decrease in absorbance from 2 to 20 s at 475 nm spectrophotometrically. Compounds were dissolved in Me₂SO at various concentrations and added to the cuvette with the MIF prior to the addition of the L-dopachrome solution.

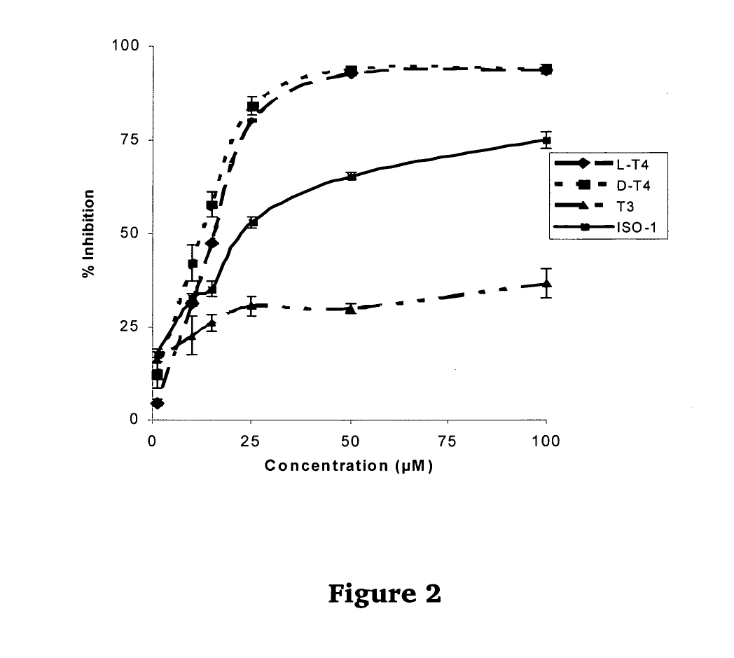

D-T4 inhibited MIF tautomerase activity in a dose-dependent manner with an IC₅₀ of 12.3 μM, which is similar to L-T4 (IC₅₀=15.8 μM) (FIG. 2). Although T4 and T3 are highly connected biologically, T3 is a weak inhibitor of MIF, which underscores the specificity of D-T4 and L-T4 as well as the biological relevance of such binding. Additionally, ISO-1 was less potent than D-T4 with an IC₅₀ value of 21 μM (FIG. 2). These data are the average of four separate experiments, and each one was carried out in triplicate.

### D-T4 is Protective in a Cecal Ligation and Puncture (CLP) Model of Sepsis

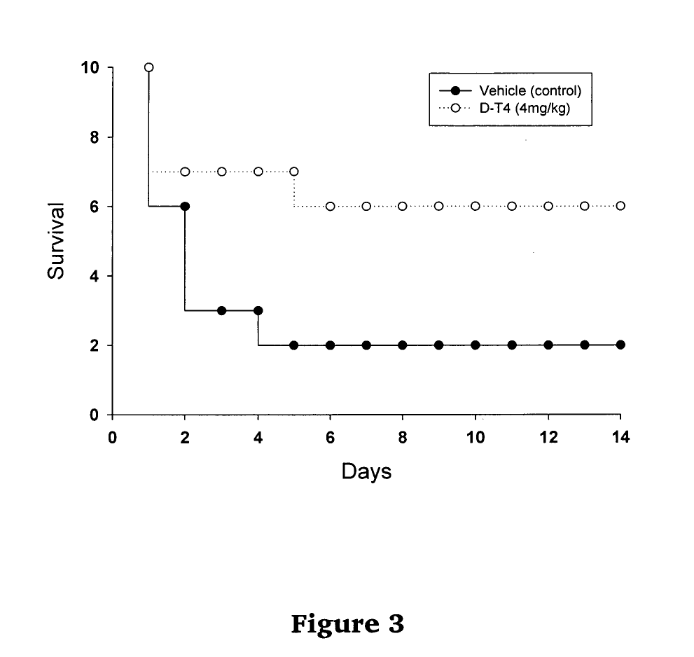

In anesthetized male Balb/C mice (ketamine 100 mg/kg and xylazine 8 mg/kg administered intramuscularly), abdominal access was gained via a midline incision. The cecum was isolated and ligated with a 6-0 silk ligature below the ileocecal valve, and the cecum punctured once with a 22 G needle, stool (approximately 1 mm³) extruded from the hole, and the cecum placed back into the abdominal cavity. The abdomen was closed with two layers of 6-0 Ethilon sutures. Antibiotics were administered immediately after CLP (Premaxin 0.5 mg/kg, subcutaneously, in a total volume of 0.5 ml/mouse) and a single dose of resuscitative fluid (normal saline solution) was administered subcutaneously (20 ml/kg-body weight) immediately after CLP. Mice were injected intraperitoneally with D-T4 (∘; 4 mg/kg, n=10) or (20 mg/kg, n=10) or vehicle (, n=10) 24 hours after CLP (FIG. 3). Additionally, two injections were given on day 2 and day 3. D-T4 (4 mg/kg) conferred protection and improved survival (6/10) compared to vehicle (2/10).

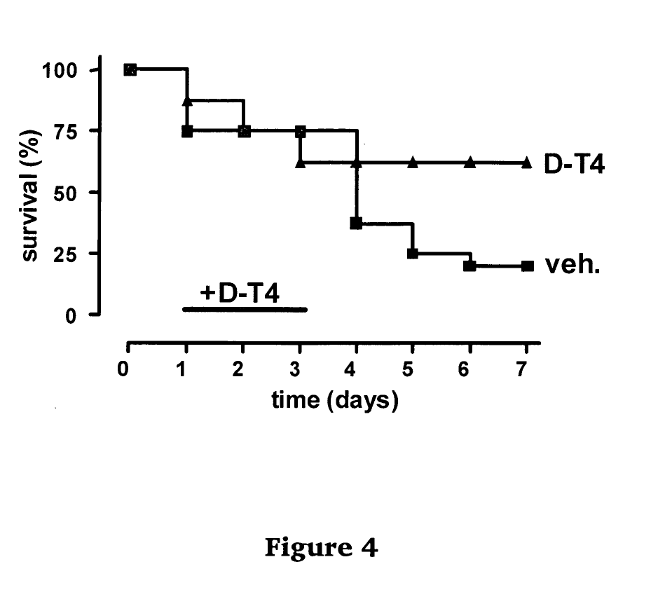

The cecal ligation and puncture experiment was repeated in a second group of animals. The experimental procedure was identical to that used in the preceding example. The results confirm that D-T4 dramatically increases survival in this model (FIG. 4).

D-T4 is a Potent Inhibitor of Tumor Necrosis Factor (TNF) Secretion from Lipopolysaccharide (LPS)-Stimulated RAW Macrophages

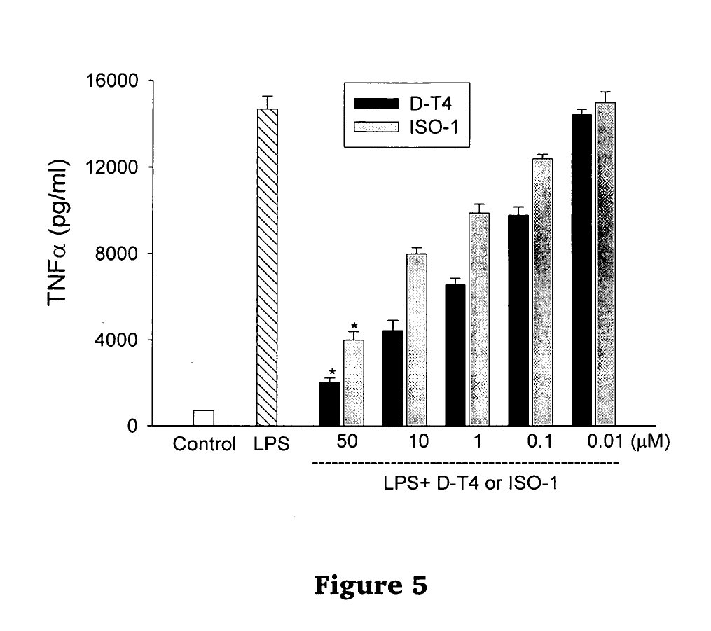

Before the addition of 0.1 μg/ml LPS (Escherichia coli 0111:B4, Sigma), 1×10⁶ RAW 267.4 cells/well were preincubated for 30 minutes with different concentration (0.01-50 μM) of D-T4 or ISO-1. After 16 h, cell culture supernatants were collected for determination of TNF concentration by enzyme-linked immunosorbent assay (R & D Systems). D-T4 is a potent inhibitor of the release of TNFα with an IC₅₀ of 1 μM (FIG. 5). Of note, T4's inhibitory effect is more potent than ISO-1 correlating with its potency in binding to the MIF active site.

### D-T4 Reduces an Inflammatory Cytokine Cascade-Induced Inflammatory Response in Wild-Type but not in MIF Knockout Mice

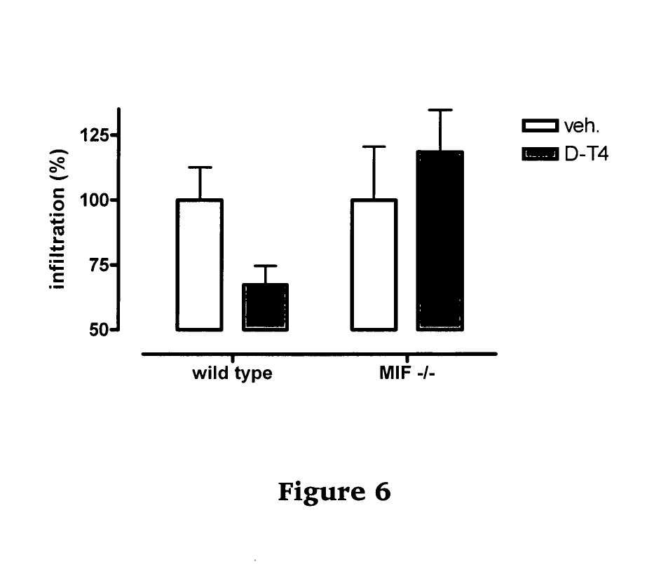

In an established model of acute inflammation (air pouch), D-T4 inhibits leukocyte recruitment in wild-type but not in MIF knockout animals. Air pouches were produced according to standard procedures (Garcia-Ramallo et al., 2002) using C57bk6 mice (wild-type) or strain-matched mice lacking both copies of the MIF gene (MIF−/−) by injecting sterile air s.c. on day 0 (6 ml) and day 3 (3 ml). On day 6, animals were treated with vehicle (350 μl of vehicle) or D-T4 (4 mg/kg) intraperitoneal (i.p.) as indicated. 15 min. later the animals were challenged by injecting 1 ml 1% carrageenan (in PBS) into the air pouch cavity. Five hours after carrageenan injection the animals were sacrificed, the pouches washed with PBS, exudates collected, and the total number of infiltrating cells quantitated. The plot in FIG. 6 shows the number of infiltrating cells normalized as a percent of the corresponding animals that received vehicle alone (veh.). *p<0.04 relative to vehicle alone; n.s., not significant. The data indicate that the mechanism of action of D-T4 operates on the MIF limb of the inflammatory cytokine cascade.

### REFERENCES

Abraham E: Why immunomodulatory therapies have not worked in sepsis. Intensive Care Med 25:556-566, 1999.
Al-Abed Y, Dabideen D, Aljabari B, Valster A, Messmer D, Ochani M, Tanovic M, Ochani K, Bacher M, Nicoletti F, Metz C, Pavlov V A, Miller E J, Tracey K J. ISO-1 binding to the tautomerase active site of MIF inhibits its pro-inflammatory activity and increases survival in severe sepsis. J Biol Chem 280:36541-36544, 2005.
Angus D C, Linde-Zwirble W T, Lidicker J, Clermont G, Carcillo J, Pinsky M R: Epidemiology of severe sepsis in the United States: analysis of incidence, outcome, and associated costs of care. Crit. Care Med 29:1303-1310, 2001.
Baue A E, Gunther B, Hartl W, Ackenheil M, Heberer G: Altered hormonal activity in severely ill patients after injury or sepsis. Arch Surg 119:1125-1132, 1984.
Beishuizen A, Thijs L G, Haanen C, Vermes I: Macrophage migration inhibitory factor and hypothalamo-pituitary-adrenal function during critical illness. J Clin Endocrinol Metab 86:2811-2816, 2001.
Beisswenger P J, Makita Z, Curphey T J, Moore L L, Jean S, Brinck-Johnsen T, Bucala R, Vlassara H: Formation of immunochemical advanced glycosylation end products precedes and correlates with early manifestations of renal and retinal disease in diabetes. Diabetes 44:824-829, 1995.
Benvenga S, Cahnmann H J, Gregg R E, Robbins J. Characterization of the binding of thyroxine to high density lipoproteins and apolipoproteins A-I. J. Clin. Endocrinol. Metab. 68:1067-1072, 1989.
Benvenga S, Cahnmann H J, Rader D, Kindt M, Robbins J: Thyroxine binding to the apolipoproteins of high density lipoproteins HDL2 and HDL3. Endocrinology 131:2805-2811, 1992.
Bernhagen J, Calandra T, Mitchell R A, Martin S B, Tracey K J, Voelter W, Manogue K R, Cerami A, Bucala R: MIF is a pituitary-derived cytokine that potentiates lethal endotoxaemia. Nature 365:756-759, 1993.
Bozza M, Satoskar A R, Lin G, Lu B, Humbles A A, Gerard C, David J R: Targeted disruption of migration inhibitory factor gene reveals its critical role in sepsis. J Exp Med 189:341-346, 1999.
Brent G A, Hershman J M: Thyroxine therapy in patients with severe nonthyroidal illnesses and low serum thyroxine concentration. J Clin Endocrinol Metab 63:1-8, 1986.
Calandra T: Pathogenesis of septic shock: implications for prevention and treatment. J Chemother 13 Spec No 1:173-180, 2001.
Calandra T, Bernhagen J, Metz C N, Spiegel L A, Bacher M, Donnelly T, Cerami A, Bucala R: MIF as a glucocorticoid-induced modulator of cytokine production. Nature 377:68-71, 1995.
Calandra T, Bucala R: Macrophage migration inhibitory factor (MIF): a glucocorticoid counter-regulator within the immune system. Crit. Rev Immunol 17:77-88, 1997.
Calandra T, Echtenacher B, Roy D L, Pugin J, Metz C N, Hultner L, Heumann D, Mannel D, Bucala R, Glauser M P. Protection from septic shock by neutralization of macrophage migration inhibitory factor. Nat Med 6:164-170, 2000.
Chagnon F, Metz C N, Bucala R, Lesur O. Endotoxin-induced myocardial dysfunction: effects of macrophage migration inhibitory factor neutralization. Circ Res 96:1095-1102, 2005.
Cvetkovic I, Al-Abed Y, Miljkovic D, Maksimovic-Ivanic D, Roth J, Bacher M, Lan H Y, Nicoletti F, Stosic-Grujicic S: Critical role of macrophage migration inhibitory factor activity in experimental autoimmune diabetes. Endocrinology 146:2942-2951, 2005.
Dios A, Mitchell R A, Aljabari B, Lubetsky J, O'Connor K, Liao H, Senter P D, Manogue K R, Lolis E, Metz C, Bucala R, Callaway D J, Al-Abed Y: Inhibition of MIF bioactivity by rational design of pharmacological inhibitors of MIF tautomerase activity. J Med Chem 45:2410-2416, 2002.
Donnelly S C, Haslett C, Reid P T, Grant I S, Wallace W A, Metz C N, Bruce L J, Bucala R: Regulatory role for macrophage migration inhibitory factor in acute respiratory distress syndrome. Nat Med 3:320-323, 1997.
Garcia-Ramallo E, Marques T, Prats N, Beleta J, Kunkel S L, Godessart N. Resident cell chemokine expression serves as the major mechanism for leukocyte recruitment during local inflammation. J. Immunol. 169(11):6467-73, 2002.
Garner L B, Willis M S, Carlson D L, DiMaio J M, White M D, White D J, Adams G A t, Horton J W, Giroir B P. Macrophage migration inhibitory factor is a cardiac-derived myocardial depressant factor. Am J Physiol Heart Circ Physiol 285:H2500-2509, 2003.
Goncalves E, Lakshmanan M, Pontecorvi A, Robbins J. Thyroid hormone transport in a human glioma cell line. Mol Cell Endocrinol. 69 (2-3):157-65. 1990.
Ivan M, Koyuncu A, Aydin C, Turan M, Gokgoz S, Sen M: Thyroid hormone supplementation in sepsis: an experimental study. Surg Today 33:24-29, 2003.
Kato Y, Muto T, Tomura T, Tsumura H, Watarai H, Mikayama T, Ishizaka K, Kuroki R: The crystal structure of human glycosylation-inhibiting factor is a trimeric barrel with three 6-stranded beta-sheets. Proc Natl Acad Sci USA 93:3007-3010, 1996.
Kavok N S, Krasilnikova O A, Babenko N A. Thyroxine signal transduction in liver cells involves phospholipase C and phospholipase D activation. Genomic independent action of thyroid hormone. BMC Cell Biol. 2001; 2:5. Epub 2001 Apr. 2.
Larsen P R: Regulation of thyrotropin secretion by 3,5,3′-triiodothyronine and thyroxine. Prog Clin Biol Res 74:81-93, 1981a.
Larsen P R, Silva J E, Kaplan M M: Relationships between circulating and intracellular thyroid hormones: physiological and clinical implications. Endocr Rev 2:87-102, 1981b.
Lawrence W D, Schoenl M, Davis P J. Stimulation in vitro of rabbit erythrocyte cytosol phospholipid-dependent protein kinase activity. A novel action of thyroid hormone. J Biol. Chem. 264(9):4766-8, 1989.
Leon-Sanz M, Lorente J A, Larrodera L, Ros P, Alvarez J, Esteban A E, Landin L: Pituitary-thyroid function in patients with septic shock and its relation with outcome. Eur J Med Res 2:477-482, 1997.
Lin H-Y, Thacore H R, Davis P J, Davis F B. Thyroid hormone potentiates the antiviral action of interferon-gamma in cultured human cells. J Clin Endocrinol Metab. 79:62-5, 1994.
Lin H-Y, Thacore H R, Davis F B, Davis P J. Thyroid hormone analogues potentiate the antiviral action of interferon-gamma by two mechanisms. J Cell Physiol. 167(2):269-76, 1996.
Lin X, Sakuragi T, Metz C N, Ojamaa K, Skopicki H A, Wang P, Al-Abed Y, Miller E J. Macrophage migration inhibitory factor within the alveolar spaces induces changes in the heart during late experimental sepsis. Shock 24:556-563, 2005.
Little J S. Effect of thyroid hormone supplementation on survival after bacterial infection. Endocrinol. 117:1431-1435, 1985.
Lolis E, Bucala R: Crystal structure of macrophage migration inhibitory factor (MIF), a glucocorticoid-induced regulator of cytokine production, reveals a unique architecture. Proc Assoc Am Physicians 108:415-419, 1996.
Lubetsky J B, Dios A, Han J, Aljabari B, Ruzsicska B, Mitchell R, Lolis E, Al-Abed Y. The tautomerase active site of macrophage migration inhibitory factor is a potential target for discovery of novel anti-inflammatory agents. J Biol Chem 277:24976-24982, 2002.
Lubetsky J B, Swope M, Dealwis C, Blake P, Lolis E: Pro-1 of macrophage migration inhibitory factor functions as a catalytic base in the phenylpyruvate tautomerase activity. Biochemistry 38:7346-7354, 1999.
Lue H, Kleemann R, Calandra T, Roger T, Bernhagen J: Macrophage migration inhibitory factor (MIF): mechanisms of action and role in disease. Microbes Infect 4:449-460, 2002.
Martin G S, Mannino D M, Eaton S, Moss M. The epidemiology of sepsis in the United States from 1979 through 2000. N Engl J Med 348:1546-1554, 2003.
Mendel C M: The free hormone hypothesis: a physiologically based mathematical model. Endocr Rev 10:232-274, 1989.
Monig H, Arendt T, Meyer M, Kloehn S, Bewig B: Activation of the hypothalamo-pituitary-adrenal axis in response to septic or non-septic diseases—implications for the euthyroid sick syndrome. Intensive Care Med 25:1402-1406, 1999.
Neves F A R, Cavalieri R R, Simeoni L A, Gardner D G, Baxter J D, Scharschmidt B F, Lomri N, Ribeiro R C. Thyroid hormone export varies among primary cells and appears to differ from hormone uptake. Endocrinology 143:476-483, 2002.
Nicoletti F, Creange A, Orlikowski D, Bolgert F, Mangano K, Metz C, Di Marco R, Al Abed Y: Macrophage migration inhibitory factor (MIF) seems crucially involved in Guillain-Barre syndrome and experimental allergic neuritis. J Neuroimmunol 168:168-174, 2005.
Phillips R H, Valente W A, Caplan E S, Connor T B, Wiswell J G: Circulating thyroid hormone changes in acute trauma: prognostic implications for clinical outcome. J Trauma 24:116-119, 1984.
Pizzagalli F, Hagenbuch B, Stieger B, Klenk U, Folkers G, Meier P J. Identification of a novel human organic anion transporting polypeptide as a high affinity thyroxine transporter. Mol. Endocrinol. 16(10):2283-96, 2002.
Rosengren E, Bucala R, Aman P, Jacobsson L, Odh G, Metz C N, Rorsman H. The immunoregulatory mediator macrophage migration inhibitory factor (MIF) catalyzes a tautomerization reaction. Mol Med 2:143-149, 1996.
Sakuragi T, Lin X, Metz C, Ojamaa K, Kohn N, Al-Abed Y, Miller E J: Lung-Derived Macrophage Migration Inhibitory Factor in Sepsis Induces Cardio-Circulatory Depression. Surgical Infections 8(1):29-40, 2007.
Senter P D, Al-Abed Y, Metz C N, Benigni F, Mitchell R A, Chesney J, Han J, Gartner C G, Nelson S D, Todaro G J, Bucala R: Inhibition of macrophage migration inhibitory factor (MIF) tautomerase and biological activities by acetaminophen metabolites. Proc Natl Acad Sci USA 99:144-149, 2002.
Slag M F, Morley J E, Elson M K, Crowson T W, Nuttall F Q, Shafer R B: Hypothyroxinemia in critically ill patients as a predictor of high mortality. JAMA 245:43-45, 1981.
Subramanya H S, Roper D I, Dauter Z, Dodson E J, Davies G J, Wilson K S, Wigley D B. Enzymatic ketonization of 2-hydroxymuconate: specificity and mechanism investigated by the crystal structures of two isomerases. Biochemistry 35:792-802, 1996.
Sugimoto H, Suzuki M, Nakagawa A, Tanaka I, Fujinaga M, Nishihira J. Crystallization of rat liver macrophage migration inhibitory factor for MAD analysis. J Struct Biol 115:331-334, 1995.
Sugimoto H, Suzuki M, Nakagawa A, Tanaka I, Nishihira J: Crystal structure of macrophage migration inhibitory factor from human lymphocyte at 2.1 A resolution. FEBS Lett 389:145-148, 1996.
Sun B, Nishihira J, Yoshiki T, Kondo M, Sato Y, Sasaki F, Todo S. Macrophage migration inhibitory factor promotes tumor invasion and metastasis via the Rho-dependent pathway. Clin Cancer Res. 11(3):1050-8, 2005.
Sun H W, Bernhagen J, Bucala R, Lolis E. Crystal structure at 2.6-A resolution of human macrophage migration inhibitory factor. Proc Natl Acad Sci USA 93:5191-6, 1996.
Suzuki M, Murata E, Tanaka I, Nishihira J, Sakai M: Crystallization and a preliminary X-ray diffraction study of macrophage migration inhibitory factor from human lymphocytes. J Mol Biol 235:1141-1143, 1994.
Suzuki M, Sugimoto H, Nakagawa A, Tanaka I, Nishihira J, Sakai M. Crystal structure of the macrophage migration inhibitory factor from rat liver. Nat Struct Biol 3:259-266, 1996.
Taylor A B, Czerwinski R M, Johnson W H, Whitman C P, Hackert M L. Crystal structure of 4-oxalocrotonate tautomerase inactivated by 2-oxo-3-pentynoate at 2.4 angstrom resolution: Analysis and implications for the mechanism of inactivation and catalysis. Biochemistry 37:14692-14700, 1998.
Taylor A B, Johnson W H, Czerwinski R M, Li H S, Hackert M L, Whitman C P: Crystal structure of macrophage migration inhibitory factor-complexed with (E)-2-fluoro-p-hydroxycinnamate at 1.8 angstrom resolution: Implications for enzymatic catalysis and inhibition. Biochemistry 38:7444-7452, 1999.
Willis M S, Carlson D L, Dimaio J M, White M D, White D J, Adams G A t, Horton J W, Giroir B P. Macrophage migration inhibitory factor mediates late cardiac dysfunction after burn injury. Am J Physiol Heart Circ Physiol 288:H795-804, 2005.
Yamamoto T, Nozaki A, Shintani S, Ishikura S, Katagiri Y, Hara A. Structure-specific effects of thyroxine analogs on human liver 3 alpha-hydroxysteroid dehydrogenase. J. Biochem. 128(1):121-8, 2000.
Yan Z, Hinkle P M. Saturable, stereospecific transport of 3,5,3′-triiodo-L-thyronine and L-thyroxine into GH4C1 pituitary cells. J Biol. Chem. 268(27):20179-84, 1993.
Yosha S, Fay M, Longcope C, Braverman L E. Effect of D-thyroxine on serum sex hormone binding globulin (SHBG), testosterone, and pituitary-thyroid function in euthyroid subjects. J Endocrinol Invest. 7(5):489-94, 1984.
PCT International Publication Number WO 2002/100332, published Dec. 19, 2002, Isoxazoline Compounds Having MIF Antagonist Activity.

1. A method for treating sepsis and/or septic shock in a subject comprising administering dextrothyroxine (D-T4) to the subject in an amount effective to treat sepsis and/or septic shock.

2. The method of claim 1, wherein the treatment inhibits macrophage migration inhibitory factor (MIF).

3. The method of claim 1, wherein the treatment prevents or reduces serum elevation of TNF-α.

4. The method of claim 1, wherein the treatment prevents or reduces tissue and/or organ injury in the subject.

5. The method of claim 1, wherein the treatment prevents or reduces septic shock.

6. The method of claim 1, wherein the treatment improves survival of the subject.

7. A method of treating a subject having a condition or disease in which it is desirable to inhibit macrophage migration inhibitory factor (MIF), the method comprising administering to the subject an amount of dextrothyroxine (D-T4) effective to inhibit MIF.

8. The method of claim 7, wherein the mammal has or is at risk for a condition or disease that comprises an inflammatory cytokine cascade that is at least partially mediated by MIF.

9. The method of claim 8, wherein the condition or disease is proliferative vascular disease; acute respiratory distress syndrome; cytokine-mediated toxicity; psoriasis; interleukin-2 toxicity; appendicitis; peptic, gastric and/or duodenal ulcer; peritonitis; pancreatitis; ulcerative, pseudomembranous, acute and ischemic colitis; diverticulitis; epiglottitis; achalasia; cholangitis; cholecystitis; hepatitis; inflammatory bowel disease; Crohn's disease; enteritis; Whipple's disease; asthma; allergy; anaphylactic shock; immune complex disease; organ ischemia; reperfusion injury; organ necrosis; hay fever; sepsis; septicemia; endotoxic shock; cachexia; hyperpyrexia; eosinophilic granuloma; granulomatosis; sarcoidosis; septic abortion; epididymitis; vaginitis; prostatitis; urethritis; bronchitis; emphysema; rhinitis; cystic fibrosis; pneumonitis; alvealitis; bronchiolitis; pharyngitis; pleurisy; sinusitis; influenza; respiratory syncytial virus infection; herpes infection; HIV infection; hepatitis B virus infection; hepatitis C virus infection; disseminated bacteremia; Dengue fever; candidiasis; malaria; filariasis; amebiasis, hydatid cysts, burns, dermatitis, dermatomyositis, sunburn, urticaria, warts, wheals; vasulitis; angiitis; endocarditis; arteritis; atherosclerosis; thrombophlebitis; pericarditis; myocarditis; myocardial ischemia; periarteritis nodosa; rheumatic fever; Alzheimer's disease; coeliac disease; congestive heart failure; meningitis; encephalitis; multiple sclerosis; cerebral infarction; cerebral embolism; Guillame-Barre syndrome; neuritis; neuralgia; spinal cord injury; paralysis; uveitis; arthritides; arthralgias; osteomyelitis; fasciitis; Paget's disease; gout; periodontal disease; rheumatoid arthritis; synovitis; myasthenia gravis; thryoiditis; systemic lupus erythematosus; Goodpasture's syndrome; Behcets's syndrome; allograft rejection; graft-versus-host disease; ankylosing spondylitis; type 1 diabetes; type 2 diabetes; Berger's disease; Retier's syndrome or Hodgkins disease.

10. The method of claim 7, wherein the subject has or is at risk for an autoimmune disease.

11. The method of claim 10, wherein the autoimmune disease is multiple sclerosis, systemic lupus erythematosus, rheumatoid arthritis, graft versus host disease, autoimmune pulmonary inflammation, autoimmune encephalomyelitis, Guillain-Barre syndrome, autoimmune thyroiditis, insulin dependent diabetes mellitus, Crohn's disease, scleroderma, psoriasis, Sjögren's syndrome or autoimmune inflammatory eye disease.

12. The method of claim 7, wherein the subject has a tumor.

13. The method of claim 7, wherein the subject has or is at risk for developing inflammation.

14. A method for reducing the pathogenic consequences of an inflammatory condition or an inflammatory cytokine cascade or for treating an inflammatory disease or condition in a subject comprising administering dextrothyroxine (D-T4) to the subject in an amount effective to reduce the pathogenic consequences of an inflammatory condition or an inflammatory cytokine cascade or to treat an inflammatory disease or condition.

15. A pharmaceutical composition comprising dextrothyroxine (D-T4) formulated in dosage form for (i) treating sepsis and/or septic shock, (ii) treating a condition or disease in a subject in which it is desirable to inhibit macrophage migration inhibitory factor (MIF), or (iii) reducing the pathogenic consequences of an inflammatory condition or an inflammatory cytokine cascade or for treating an inflammatory disease or condition.

16. A method of preparing the pharmaceutical composition of claim 15 for treating sepsis and/or septic shock, the method comprising formulating dextrothyroxine (D-T4) in a pharmaceutical composition in an amount effective to (i) treat sepsis and/or septic shock, (ii) to inhibit MIF, or (iii) to reduce the pathogenic consequences of an inflammatory condition or an inflammatory cytokine cascade or treat an inflammatory disease or condition.

17-20. (canceled)
